(19) United States
(12) Patent Application Publication
(10) Pub. No.: US 2006/0249593 A1
(43) Pub. Date: Nov. 9, 2006

(54) Dispensing device and method

(75) Inventors: Colin Brown, Egham, Surrey (GB); Kishen Gohil, New Malden, Surrey (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 10/554,344

(22) PCT Filed: Apr. 28, 2004
(86) PCT No.: PCT/CH04/00260

§ 371 Date: Oct. 24, 2005

(30) Foreign Application Priority Data
Apr. 30, 2003 (GB) 03097789

(51) Int. Cl. A61L 9/12; A61L 9/04

(52) U.S. Cl. 239/44; 422/124; 239/45

(57) ABSTRACT

A dispensing device for dispensing a volatile material to an ambient environment comprising: a refill containing a volatile liquid, a porous wick having a lower portion extending within the refill and adapted to be in fluid communication with said volatile liquid within the reservoir and an upper portion extending outside of the reservoir and adapted to be in fluid communication with an ambient environment, and a shaped recess within the upper portion of the porous wick, and further, housing element adapted to engage the shaped recess in the upper portion of the porous wick when the dispensing device is assembled. The dispensing device denies the use of inappropriate refills in the device. The dispensing device is particularly useful in dispensing volatile materials such as fragrances, odor masking agents, insecticides, medicaments as well as other volatile materials which may have a cosmetic, insecticidal or medical effect to an ambient environment.

The present invention relates to improved dispensing devices useful for the delivery of a volatile or evaporable material to an ambient environment, such as a room, or interior of a vehicle.

There are known in the art various devices useful for the delivery of volatile materials such as fragrances, odor-masking agents, insecticides, medicaments and other volatile materials, which may have a cosmetic, insecticidal or medical effect. One particular class of such devices includes those that are used for the delivery of a liquid volatile or evaporable composition to an ambient environment. A typical device includes a reservoir or other container capable of containing an amount of a volatile material, from which reservoir protrudes a wick. The wick operates to transport the liquid by means of capillary action from the interior of the reservoir to the ambient environment, into which it evaporates or volatilizes. Such devices are simple, and frequently effective. In certain embodiments, such devices are supplied as part of a larger apparatus, which may provide an external feature for static diffusion systems or it may include a means of diffusing the volatile liquid, such as a heat source, which may surround some or part of the wick, and when heated induces more rapid volatilization of the liquid, or a fan that causes more rapid volatilization of the liquid into the ambient environment.

Such devices may be of a single-use type, to be discarded after use, or they may be of a multi-use device wherein the consumer replaces only a refill in the dispensing device when the supply of volatile material has been exhausted. The latter permits the consumer to reuse the dispensing device, and indeed provides flexibility in its use as often various different volatile liquids may be available for use, and the consumer can select among these those which are most appropriate or most favored for use.

While such devices are often effective, they are nonetheless not without shortcomings. One primary concern resides in the fact that the use of inappropriately-dimensioned refills in dispensing devices provide a real risk of malfunction, which may be annoying to the consumer, and may also be dangerous in certain instances. The manufacture of dispensing devices requires precision in their design and assembly, and when of the multi-use type, the use of appropriate refills is important to their optimal performance. Such inappropriate refills may, for instance, be refills produced by an unauthorized supplier, which may not meet the specific dimensions or the specific quality of appropriate refills that are specifically designed and produced for use in the specific dispensing devices. While the use of inappropriately-dimensioned refills is to be avoided due to the risk of malfunction, nonetheless consumers may inadvertently or inappropriately seek to use inappropriate refills in multi-use type of dispensing devices.

Accordingly, there is a real and urgent need in the art for improved devices useful for the delivery of volatile materials such as fragrances, odor-masking agents, insecticides, medicaments as well as other volatile materials, which improved devices provide a means for preventing the use of inappropriate refills in a dispensing device.

In one aspect the present invention provides a dispensing device for dispensing a volatile material to an ambient environment comprising:

a refill, which includes a reservoir containing a volatile liquid, and a porous wick having a lower portion extending within the reservoir and adapted to be in fluid communication with said volatile liquid within the reservoir and an upper portion extending outside of the reservoir and adapted to be in fluid communication with an ambient environment, wherein the upper portion of the porous wick has a shaped recess; and,

a housing element adapted to engage the shaped recess in the upper portion of the porous wick when the dispensing device is assembled.

A still further aspect of the invention is directed to a process for preventing the use of inappropriate refills in a dispensing device, comprising the steps, providing
a) a refill device comprising a shaped recess in the upper portion of a porous wick, and
b) a housing element adapted to engage the shaped recess when the dispensing device is assembled.

These and other aspects of the invention will become apparent from the following specification and figures.

FIG. 1 depicts a cross-sectional schematic view of the cover portion of a dispensing device.

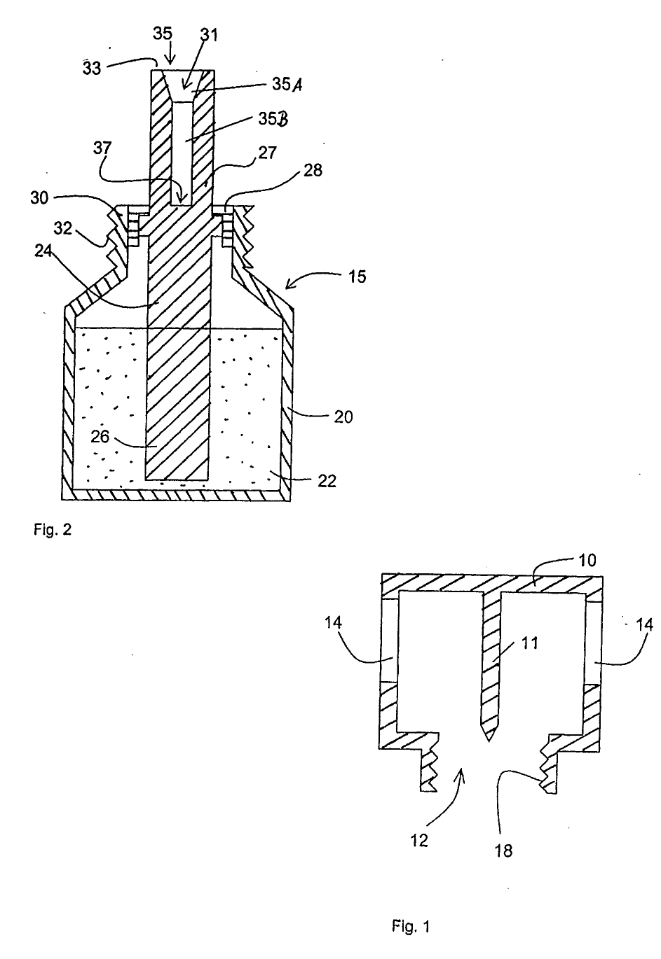

FIG. 2 illustrates a cross-sectional schematic view of the refill portion of a dispensing device according to the invention.

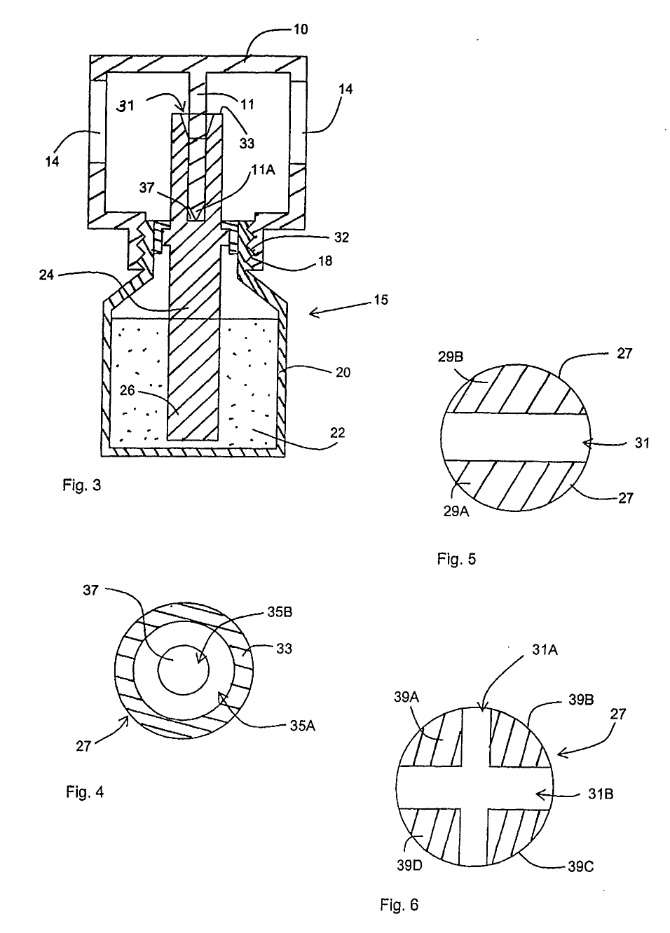

FIG. 3 illustrates a cross-sectional schematic view of the cover portion depicted on FIG. 1 and the refill portion depicted on FIG. 2 assembled to form a dispensing device 5 according to the invention.

FIG. 4 shows a schematic view from the end of the wick according to the inventive embodiment illustrated on FIGS. 2 and 3.

FIG. 5 illustrates a cross-sectional schematic view of an alternative embodiment of the wick according to the invention.

FIG. 6 illustrates a cross-sectional schematic view of a further alternative embodiment of the wick according to the invention.

The invention provides a means for preventing the use of inappropriate refills in a dispensing device by providing a shaped recess in the upper portion of the porous wick forming a part of the refill device and a housing element adapted to engage the shaped recess in the upper portion of the porous wick when the dispensing device is assembled.

FIG. 1 depicts a cross-sectional schematic view of a cover 10 portion of a dispensing device, including a housing element 11 adapted to engage a shaped recess in the upper portion of the porous wick when the dispensing device is assembled. The cover 10 has an inlet 12 adapted to receive a portion of a reservoir containing a volatile liquid, and further includes two vent holes 14, which permits volatilized liquid to evaporate from within the interior of the cover 10. In the embodiment shown in FIG. 1, the inlet 12 includes a series of threads 18 dimensioned to provide a mating fit with corresponding threads present on the reservoir of a refill (not shown). While two vent holes 14 are depicted, a lesser or greater number may be included in the cover 10. Indeed, in the simplest form of the invention, no cover is required, and any arrangement of elements or parts which provides for a housing element 11 adapted to engage the shaped recess in the upper portion of the porous wick when the dispensing device is assembled is sufficient. The said housing element may be associated with a different part of the dispensing device other than a cover as shown here and still fall within the inventive teaching.

FIG. 2 depicts in cross-section a schematic view of a refill 15 of a dispensing device according to the invention. The refill 15 includes a reservoir 20 containing a volatile liquid 22, and a porous wick 24 having a lower portion 26 extending within the reservoir 20 and in fluid communication with the volatile liquid 22, and an upper portion 27 extending outside of the reservoir and adapted to be in fluid communication with an ambient environment. In the embodiment shown, the porous wick 24 is retained in the reservoir 20 by an annular fitment 28 within a neck 30 of the reservoir 20. The neck 30 includes exterior threads 32, which are dimensioned to mate with corresponding threads 18 of the cover 10 (see FIG. 1). The upper portion 27 of the porous wick 24 further includes a shaped recess 31 extending downwardly from the top end 33 of the porous wick 24. The shaped recess 31 defines a cavity having an open end 35 and a bottom end 37 within the upper portion 27 of the porous wick 24. In the embodiment shown, the shaped recess 31 includes a first tapered region 35A adjacent to the top end 33, and a cylindrical region 35B extending downwardly from the tapered region 35A and terminating at the bottom end 37. In the embodiment shown, the total height of the shaped recess 31 extends concentrically within the wick 24 from the end 33 thereof to about the level of the fitment 28; however different total heights and indeed different configurations for the shaped recess 31 may be practised within the scope of the present invention. It is only required that, when the refill 15 is assembled into the dispensing device, at least a portion of the housing element 11 (see FIG. 1) engages the shaped recess 31 in the porous wick 24 when the dispensing device is assembled.

FIG. 3 illustrates a cross-sectional schematic view of the cover portion depicted on FIG. 1 and the refill portion depicted on FIG. 2 assembled to form a dispensing device according to the invention. The dispensing device is assembled by inserting the neck 30 (see FIG. 1) of the reservoir 20 into the inlet 12 (see FIG. 1) of the cover 10, and rotating the corresponding mating threads 18, 32 to removably assemble the cover 10 with the reservoir 20 and in so doing, simultaneously engage the portion of the housing element 11 within the shaped recess 31 in the porous wick 24. Where the shaped recess 31 includes a first tapered region 35A (see FIG. 2) adjacent to the top end 33, entry of the end 11A of the housing element 11 is facilitated, as the tapered sidewalls aid in guiding the housing element 11, particularly if the end 11A is also tapered as shown in FIGS. 1 and 3. When assembled, the length of the housing element 11 is sufficient such that the end 11A is adjacent to the bottom end 37 however such is not essential.

It is to be understood that, while mating screw threads are discussed and depicted on FIGS. 1, 2 and 3, the method of insertion can involve other techniques of assembly including, e.g., snapping the refill into place in a cavity having features that engage the thread, such as an extended pin or flexible tab, as well as snapping the refill in place in a larger cavity that engages features on the refill itself, other than on the neck.

As will be understood from the foregoing and from FIGS. 2 and 3, the shaped recess in the upper portion of the porous wick forming a part of the refill cooperates with an appropriately-dimensioned and appropriately-positioned housing element adapted to engage the shaped recess when the dispensing device is assembled. The use of an inappropriately-shaped recess in the wick of the refill prevents a consumer inserting and assembling a dispensing device, thus preventing its use. While the embodiment shown in and described in FIGS. 2 and 3 illustrate a shaped recess 31 having a generally circular cross-section and a housing element 11 (see FIG. 1) which is also generally circular in cross-section, any other shape may be used for shaped recess 31 and/or the housing element 11. The use of a plurality of shaped recesses 31 and/or a plurality of housing elements 11 is also possible.

FIG. 4 shows a plan view of the end of the wick according to the inventive embodiment illustrated on FIGS. 2 and 3. In this view, end 33 of the upper portion 27 of the porous wick is visible, as well as the tapered region 35A adjacent to the top end 33, and a cylindrical region 35B extending downwardly from the tapered region 35A and terminating at the bottom end 37, all of which are concentric with the central axis of the porous wick

FIG. 5 illustrates a plan view of an upper portion 27 of a porous wick in an alternative embodiment of the wick according to the invention. Herein the recess 31 is a channel cut through the upper portion 27 of the porous wick, dividing it into two sections, 29A and 29B. While not illustrated, such an embodiment of the upper portion 27 of the porous wick and recess 31 is particularly suited for use with a housing element having a planar geometry. Such elements are well suited for use with a snap-in type of refill.

FIG. 6 illustrates a plan view of an upper portion 27 of a porous wick in a further alternative embodiment of the wick according to the invention. Herein the recess is defined by two intersecting channels 31A, 31B cut through the upper portion 27 of the porous wick, dividing it into four sections, 39A, 39B, 39C and 39D. While not illustrated, such an embodiment of the upper portion 27 of the porous wick and recess 31 is particularly suited for use with a housing element having two intersecting planar plates or parts, which define a cross. Such elements are well suited for use with a snap-in type of refill.

With regard to the various elements of the dispensing devices according to the present invention, any material that can be suitably formed or shaped into forms appropriate to fulfilling the functions described above can be used. For example, for the reservoir 20 and fitment 28 of the refill 15, suitable materials are non-porous materials which, by way of non-limiting example, include glass, ceramic materials, metals, as well as various polymers including high density polyolefins especially high density polyethylene, polyalkylene terephthalates, such as polyethylene terephthalate, polyvinyl chloride, Berex(R), as well as other materials that are not deleteriously affected by the volatile liquid. Desirably the reservoir is formed of a clear or translucent material, which may optionally be colored, such as a glass or suitable polymer. Polymeric materials are particularly suited for forming the fitment, as they are typically readily moldable, but other materials may be used instead. The reservoir itself may be of any shape, form or configuration that is suited to the containment of a volatile liquid that is to be provided therein. Thus, various shapes and configurations based on aesthetic reasons having pleasing forms and shapes may be produced.

With regard to the porous wick that is included in the dispensing devices according to the present invention, the porous wick may be formed of materials known to the art. Such materials include, by way of non-limiting example, those based on natural or synthetic fibers, woven or non-woven fabrics, cords, rods or other articles, porous media such as those based on celluloses such as cardboard-based and paper-based wicks, graphite and carbonaceous wicks, as well as porous synthetic polymers and porous ceramic materials. Such materials are commercially available from a variety of sources, including porous synthetic polymers from Porex Corp., (Fairburn, Ga., USA) and Micropore Plastics Inc. (Stone Mountain, Ga.) as well as porous ceramics available from Rauschert GmbH & Co.-KG (Germany). With regard to the porous wick, it is only required that it be capable of absorbing the volatile liquid 22 in the reservoir 20, and transport it by means of capillary action. The shape, of the wick itself, particularly its cross-sectional shape may be of any shape, form or configuration and is not limited to the form shown in the Figures, which is a porous wick having a circular cross-sectional shape. For example, a porous wick having those sidewalls is useful, especially when the wick is to be used in conjunction with a secondary wick.

The volatile liquid which may be used in the dispensing devices taught herein may be any volatile or evaporable material, such as fragrances, odor masking agents, insecticides, medicaments as well as other volatile materials which may have a cosmetic, insecticidal or medical effect which is known to the art. Desirably the volatile liquid is one or more liquids which have a cosmetic effect such as a fragrancing or odor masking effect such as may be based on one or more essential oils, or volatile liquids which have an insecticidal effect or a medical effect. By way of non-limiting example, useful essential oils include one or more of: anethole 20/21 natural, aniseed oil china star, aniseed oil globe brand, balsam (Peru), basil oil (India), black pepper oil, black pepper oleoresin 40/20, Bois de Rose (Brazil) FOB, borneol flakes (China), camphor oil, canaga oil (Java), cardamom oil, cassia oil (China), cedarwood oil (China), cinnamon bark oil, cinnamon leaf oil, citronella oil, clove bud oil, clove leaf, coriander (Russia), coumarin (China), cyclamen aldehyde, diphenyl oxide, ethyl vanillin, eucalyptol, eucalyptus oil, eucalyptus citriodora, fennel oil, geranium oil, ginger oil, ginger oleoresin (India), white grapefruit oil, guaiacwood oil, gurjun balsam, heliotropin, isobornyl acetate, isolongifolene, juniper berry oil, L-methyl acetate, lavender oil, lemon oil, lemongrass oil, lime oil, litsea cubeba oil, longifolene, menthol, methyl cedryl ketone, methyl chavicol, methyl salicylate, musk ambrette, musk ketone, musk xylol, nutmeg oil, orange oil, patchouli oil, peppermint oil, phenyl ethyl alcohol, pimento berry oil, pimento leaf oil, rosalin, sandalwood oil, sandenol, sage oil, clary sage, sassafras oil, spearmint oil, spike lavender, tagetes, tea tree oil, vanilin, vetyver oil (Java), wintergreen.

These and other suitable materials which may be useful in the volatile liquid may be commercially obtained from a variety of suppliers including: Givaudan Corp. (Teaneck, N.J.); Berje Inc. (Bloomfield, N.J.); BBA Aroma Chemical Div. of Union Camp Corp. (Wayne, N.J.); Firmenich Inc. (Plainsboro N.J.); Quest International Fragrances Inc. (Mt. Olive Township, N.J.); Robertet Fragrances Inc. (Oakland, N.J.), or from other suppliers not necessarily listed herein. The volatile liquids may be provided in the form of neat compositions, or may be provided as aqueous mixtures, organic mixtures or aqueous-organic mixtures which include of one or more volatile or evaporable materials.

The dispensing device according to the invention may itself be used to dispense a volatile liquid into an atmosphere. It may also be used in conjunction with a further apparatus, such as in a heated vaporizer device, wherein at least a portion of the dispensing device is heated so to controllably induce delivery of the volatile liquid to the ambient environment. The dispensing device is particularly conveniently used as a refill for said heated vaporizer device. Similarly, the dispensing device may be used in conjunction with a further apparatus such as being part of a device that induces an airflow, by means of a fan or other device, wherein at least a portion of the dispensing device, particularly a secondary wick, is inserted in the induced airflow so to controllably induce the delivery of the volatile liquid to the ambient environment.

The dispensing device according to the invention may be used to dispense a volatile liquid in any ambient environment. Non-limiting examples of ambient environments include interior spaces of buildings or other structures such as rooms, hallways, elevators, common areas, closets, as well as the interior spaces of vehicles such as automobile interiors, boat interiors, aircraft cabins, and the like.

While the invention is susceptible of various modifications and alternative forms, it is to be understood that specific embodiments thereof have been shown by way of example in the drawings which are not intended to limit the invention to the particular forms disclosed; on the contrary the intention is to cover all modifications, equivalents and alternatives falling within the scope and spirit of the invention as expressed in the appended claims.

1. A dispensing device for dispensing a volatile material to an ambient environment comprising:
a refill which includes a reservoir containing a volatile liquid, and a porous wick having a lower portion extending within the reservoir and adapted to be in fluid communication with said volatile liquid within the reservoir and an upper portion extending outside of the reservoir and adapted to be in fluid communication with an ambient environment, wherein the upper portion of the porous wick has a shaped recess;
and,
a housing element adapted to engage the shaped recess in the upper portion of the porous wick when the dispensing device is assembled.

2. A process for preventing the use of inappropriate refills in an air treatment device which comprises contemplates the step of providing:
a refill device comprising a shaped recess in the upper portion of a porous wick, and
a housing element adapted to engage the shaped recess when the dispensing device is assembled.

3. A dispensing device according to claim 1 wherein:
the shaped recess of the wick defines a cavity having an open end, and a bottom end within the upper portion of the porous wick.

4. A dispensing device according to claim 1 wherein:
the shaped recess of the wick includes a tapered region.

5. A dispensing device according to claim 4 wherein:
the tapered region of the shaped recess is adjacent to a top end of the wick.

6. A dispensing device according to claim 1 wherein:
the shaped recess of the wick defines a cavity having an open end, a tapered region adjacent to the top end of the wick, and a cylindrical region extending downwardly from the tapered region of wick.

7. A dispensing device according to claim 6 wherein:
the shaped recess terminates at a bottom end terminating the cylindrical region of the shaped recess.

8. A dispensing device according to claim 6 wherein:
the tapered region and the cylindrical region are concentric with the central axis of the wick.

9. A dispensing device according to claim 1 wherein:
the shaped recess of the wick is a channel extending through the upper portion of the wick.

10. A dispensing device according to claim 1 wherein:
the shaped recess of the wick are two intersecting channels extending through the upper portion of the wick.

11. A dispensing device according to claim 1 which further includes a fan.
